(19) United States
(12) Patent Application Publication
(10) Pub. No.: US 2015/0238128 A1
(43) Pub. Date: Aug. 27, 2015

(54) Sensor Instrument

(71) Applicant: SIEMENS AKTIENGESELLSCHAFT, München (DE)

(72) Inventors: Mario Bechtold, Hemhofen (DE); Stefan Förtsch, Erlangen (DE); Rainer Kuth, Hochstadt (DE)

(21) Appl. No.: 14/423,060

(22) PCT Filed: Jul. 19, 2013
(86) PCT No.: PCT/EP2013/065308

§ 371 Date: Feb. 20, 2015

(30) Foreign Application Priority Data
Aug. 20, 2012 (DE) 10 2012 214 737.3

(51) Int. Cl. A61B 5/1473; A61B 1/273; A61B 5/00

(52) U.S. Cl. CPC A61B 5/1473; A61B 5/4233; A61B 5/4238; A61B 5/4255; A61B 1/2736

(57) ABSTRACT

The invention relates to a sensor instrument (2) for examining the mucous membrane of the oesophagus, stomach and duodenum of a patient to see whether it is infected with bacteria. Said instrument comprises an evaluation unit (4) and a catheter probe (6) with an ammonia-sensitive sensor (8) and a catheter.

This application is the National Stage of International Application No. PCT/EP2013/065308, filed Jul. 19, 2013, which claims the benefit of German Patent Application No. DE 10 2012 214 737.3, filed Aug. 20, 2012. The entire contents of both documents are hereby incorporated herein by reference.

### BACKGROUND

The present embodiment relate to a sensor instrument.

Such a sensor instrument is described in DE 10 2010 006 969 A1, which may be traced back to the applicant.

A possible reason for discomfort of a patient in the region of the upper gastrointestinal tract is an infection with Helicobacter pylori bacteria.

DE 10 2010 006 969 A1 has disclosed a test method, with the aid of which a patient may be examined for such an infection. Use is made of a gastroscope with an insertion tube, at the distal end of which a sensor, which reacts sensitively to ammonia, is arranged. Use is made of the fact that Helicobacter pylori bacteria split urea into carbon dioxide and ammonia using the urease enzyme, and that ammonia is typically only detectable in relevant amounts in the stomach of a patient in the case of an infection with Helicobacter pylori bacteria. Therefore, the presence of an increased amount of ammonia and, as a consequence, also an infection with Helicobacter pylori bacteria may be deduced in the case of a corresponding reaction of the sensor, which is positioned in the stomach of a patient.

The basic functional principle of the sensor was presented, inter alia, within the scope of the presentation “Immediate detection of Helicobacter infection with a novel electrochemical system” (Gastroenterology, volume 138, issue 5, supplement 1, pages S-114, May 2010) by Helmut Neumann, Stefan Foertsch, Michael Vieth, Jonas Mudter, Rainer Kuth and Markus F. Neurathduring at the “DIGESTIVE DISEASE WEEK 2010”. According thereto, a change in an electric variable is registered metrologically when an electrode pair comes into contact with ammonia. One electrode of the electrode pair reacts chemically with the ammonia.

A gastroscope is a special endoscope for examining the mucous membrane of the esophagus, stomach and duodenum, and the gastroscope is therefore a relatively complex medical instrument that is produced with a relatively high technical and financial outlay. As a result of continuously increasing costs in the health sector as well, it is advantageous to configure sensor instruments for the medical field such that the production thereof may be carried out as easily and cost-effectively as possible.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a simple sensor instrument is provided.

The sensor instrument serves for examining the mucous membrane of the esophagus, stomach and duodenum of a human or animal patient to see whether the mucous membrane is infected with bacteria that, due to a corresponding metabolic reaction, emit ammonia to the surroundings thereof (e.g., also, bacteria of the genus Helicobacter, such as Helicobacter pylori, Helicobacter heilmannii or “Candidatus Helicobacter suis”). The sensor instrument is constructed as an assembly of an evaluation unit and a catheter probe including an ammonia-sensitive sensor and a catheter.

An assembly may be that the individual components form a functional unit (e.g., the components are matched to one another technically and only configured for the interaction with the other components). The sensor instrument therefore has a very simple design and is accordingly producible without major technical or financial outlay. This applies, for example, to the evaluation unit, which may only interact with the sensor and also has no freely allocatable connectors. The evaluation unit may include a simple circuit for evaluating the signals formed by the sensor element and an indication element for displaying the evaluation result, and possibly also includes a memory.

In one embodiment, the sensor instrument includes no further functional units in addition to the evaluation unit, sensor and catheter functional units. Overall, the structure follows that of, for example, a simple commercial digital clinical thermometer.

In accordance with an advantageous embodiment, the catheter probe of the sensor instrument is embodied for use in an endoscopic work channel such that the sensor instrument may be used within the scope of gastroscopy. The sensor instrument is then inserted into the work channel of the gastroscope catheter already inserted into the esophagus of a patient. Thus, in this case, the sensor instrument serves as an optionally usable probe that, just like further instruments (e.g., an instrument for biopsy) is used where necessary during gastroscopy and is inserted into the work channel of the in-use gastroscope. For example, the sensor instrument has such a configuration that the sensor instrument is suitable for and compatible with as many differently configured gastroscopes (e.g., different models from different producers) as possible.

The catheter probe may be detachably connected to the evaluation unit. In this case, the handling of the sensor instrument is simplified by virtue of, for example, the catheter probe initially being inserted into, for example, the work channel of a gastroscope, The evaluation unit, which, for example, is housed in a convenient housing, is only subsequently connected to the catheter probe and, for example, is plugged onto the catheter probe with the aid of a simple plug-in connection.

The catheter probe may be embodied as a disposable (or single-use) article that, for example, may be disposed of by way of clinical refuse. This disposable article may be enclosed in sterile packaging within the scope of production, and, for example, the disposable article is not used only a few times but rather, within the meaning of the term single-use article, is used a single time. For example, disposable articles offer the advantage that the disposable articles need not be cleaned and disinfected after use, said work steps generally being accompanied by relatively high work and time outlay.

In one development, the sensor instrument is embodied as a complete, intrinsically sealed and therefore non-dismountable (without destruction) assembly, and in addition, as a disposable article. The evaluation unit is therefore an integral, non-detachable component and may likewise be embodied as a disposable article. This article is then removed from sterile packaging when necessary, used and subsequently disposed of.

In accordance with a further embodiment, the sensor instrument also includes simple endoscopic optics. The sensor instrument is not embodied in the style of a micromechanical tool for use in an endoscopic work channel of a gastroscope, but rather, the sensor instrument is used as a type of simple disposable gastroscope. In addition to conventional gastroscopes with high quality optics and a work channel for micromechanical instruments, medical endoscopes with a very simple design are currently also being developed and used. The medical endoscopes are provided as disposable articles and disposed of after a single use. By contrast, in the case of conventional gastroscopes, these are cleaned and disinfected after use. Cleaning of the work channel, for example, is connected with relatively high outlay. In order to avoid this outlay, simplified instruments are being developed for specific application scenarios, in which, for example, a work channel is dispensed with, and the number of Bowden cables for the movement control is reduced. For example, by reducing the functions of these instruments, the production outlay is also markedly reduced, both from a technical and a financial point of view, such that these too may be considered disposable articles.

The evaluation unit may only be configured for evaluating the signals from the sensor and, for example, housed in a disinfectable plastic housing. In the case of a necessary power supply for the evaluation electronics, provision may be made for a battery. In the case of a disposable article, provision may be made for an alternative energy supply that is uncritical in terms of disposal. The evaluation unit is kept as simple as possible, as is also the case in, for example, a commercial digital clinical thermometer. By way of example, an embodiment that is substantially constituted by a circuit made of an operational amplifier, an analog/digital transducer, and a processor for balancing a characteristic is provided for value discrimination and for controlling an indicator.

Depending on the application scenario, the evaluation unit is configured to output an examination report. In this case, provision may be made for a wireless transmission of the examination report to a terminal. Therefore, the evaluation unit includes a data interface (e.g., a wireless module) as a further functional unit. The evaluation unit and the terminal are configured for wireless transmission and for receiving information. A corresponding examination report then contains, for example, information such as the result of the examination, an instrument number identifying the respective instrument, a measurement number identifying the examination, date and time of the examination, etc. Transmission protocols based on Bluetooth, WLAN or Wireless USB may be used for the wireless transmission of information between the sensor instrument and the terminal. The examination reports received by the terminal may then be directly entered into the clinical software (e.g., saved in a stored patient file).

If such data interchange is provided between sensor instruments and a terminal, the terminal may confirm the reception of an examination report, and if, as a result of a corresponding confirmation communication by the terminal, the corresponding sensor instrument, from which the examination report was sent, is put into a standby mode as a result of the confirmation message. As a result, the sensor instrument is only activated during the actual use, which, in the case of battery operation, lengthens the battery replacement cycle.

The evaluation unit and therefore the sensor instrument may include an indicator with two optical signals. One signal shows the presence of, for example, Helicobacter pylori bacteria, and one signal shows the non-presence of the bacteria. Thus, for example, a light-emitting diode is attached at the evaluation unit. The light-emitting diode lights up red if an infection with Helicobacter pylori bacteria is to be deduced, and the light-emitting diode lights up green if the findings are negative.

In an alternative or complementary manner thereto, the evaluation unit includes an acoustic signal output with two output values. One output value symbolizes the presence of corresponding bacteria, and one output value symbolizes the non-presence thereof. The corresponding output values may, for example, be realized by pulse sequences with different frequencies.

Provision is also made for signaling an active measurement to a user, either by a further output value of the acoustic signal output and/or by a further optical signal of the indicator.

### BRIEF DESCRIPTION OF THE DRAWINGS

FIG. 1 shows a side view of one embodiment of a sensor instrument including one sensor;

FIG. 2 shows a magnified illustration of the sensor; and

FIG. 3 shows a view of the sensor instrument without a lining

### DETAILED DESCRIPTION

Parts corresponding to one another have respectively been provided with the same reference signs in all figures.

The sensor element 2 described below in an exemplary manner serves for examining the mucous membrane of the esophagus, stomach and duodenum of a patient and is used within the scope of gastroscopy.

Within the scope of such a gastroscopy, a flexible endoscope or video endoscope is used for subjecting the gastrointestinal tract of the patient to a visual examination. In the case of an examination of the upper gastrointestinal tract, a tube or catheter with optical components is introduced into the mouth of the patient and subsequently gradually advanced until the desired position for the examination is reached. Within this catheter, there typically is at least one work channel for inserting micromechanical instruments, such as small forceps, such that, for example, a biopsy may be undertaken in addition to the visual examination.

If there is a suspected infection with Helicobacter pylori bacteria, testing whether an infection with these bacteria is present may be carried out in the simplest possible way within the scope of the gastroscopy. The sensor element 2, which is inserted into the work channel of the endoscope, serves this purpose.

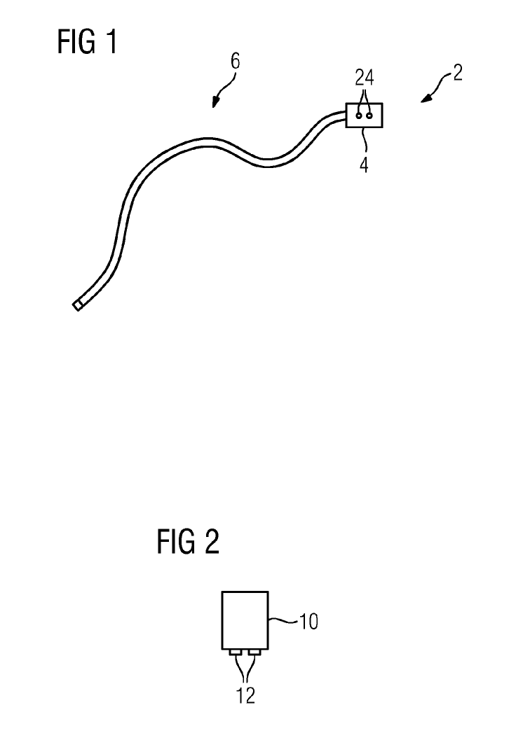

The sensor instrument 2 schematically depicted in FIG. 1 has a two-part design, where the two parts, an evaluation unit 4 and a catheter probe 6 including an ammonia-sensitive sensor 8, are connected to one another by way of a simple plug-in connection.

The catheter probe 6 is flexible and configured as a disposable article, where, prior to use, the disposable article is removed from sterile packaging and disposed of via clinical refuse after a single use. When necessary, the catheter probe 6 is inserted into the work channel of the endoscope and advanced until the end opposite to the evaluation unit 4, at which end the sensor 8 is positioned, is in contact with the mucous membrane to be examined or with the stomach contents of the patient.

In an alternative embodiment not depicted here in any more detail, endoscopic optics are integrated into the catheter probe such that the sensor instrument 2, embodied as a disposable article, has a dual function, and the path of the catheter probe 6 may be followed optically. The optical signals are transmitted to a display unit. The actuation of the integrated optical elements and the evaluation of the optical signals may be carried out in the evaluation unit 4.

At the end side, the sensor 8, depicted in a magnified manner in FIG. 2, includes two electrodes 12 embedded in a plastic casing 10, the ends of which electrodes protrude out of the plastic casing 10 and are therefore exposed. The two electrodes 12 are stainless steel wires 14, the ends of which have been provided with a different coating 16. One of the two electrodes 12 serves as a reference electrode and is coated with gold or platinum. The other electrode 12 serves as an ammonia-sensitive electrode 12 and is coated with a silver chloride layer and, lying therebelow, a silver layer.

If the two electrodes 12 lie in an electrolyte, such as the stomach contents, the two electrodes 12, together with the electrolyte, form a type of galvanic cell that, due to the silver chloride coating on one of the two electrodes 12, is initially inactive. The silver chloride layer is insoluble in water and stomach acid and prevents an ion flow such that no potential difference may be established between the two electrodes 12.

However, in the case of an infection with Helicobacter pylori bacteria, there is an increased concentration of ammonia in the stomach contents. The ammonia chemically reacts with the silver chloride. This produces a water-soluble complex such that the silver chloride layer is removed. As soon as the silver layer lying therebelow is exposed, the galvanic cell is activated, and a potential difference that may be registered metrologically is established between the electrodes 12. The metrological registration is brought about with the aid of the evaluation unit 4, which is plugged onto the catheter probe 6 for this purpose.

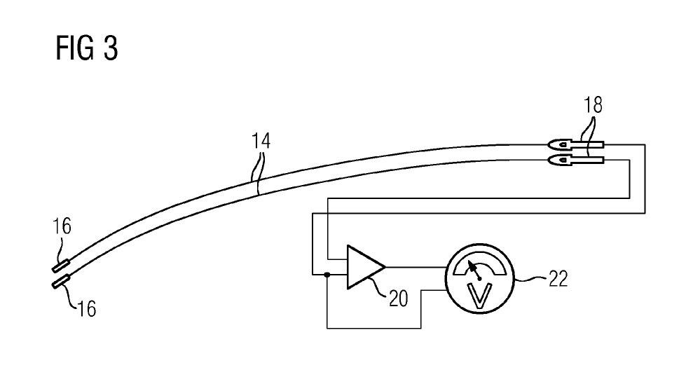

A particularly simple configuration of the sensor instrument 2 is depicted in a schematic-like manner in FIG. 3 without lining (e.g., without the plastic casing 10 and without the housing for the evaluation unit 4). Here, the stainless steel wires 14 are depicted with the coating 16 at one end, once with gold and once with silver chloride and, lying therebelow, silver. The wires 14 are connected in an electrically conducting manner to a circuit by simple plug-in contacts 18. The circuit substantially includes an amplifier 20 and a voltage measuring device 22 including an indicator.

For particularly simple handling, provision is alternatively made for equipping the evaluation unit, as depicted in FIG. 1, with an optical indicator made of two light-emitting diodes 24. In the case of a positive examination result, one of the light-emitting diodes lights up red, and in the case of a negative examination result (e.g., if there clearly is no infection with Helicobacter pylori bacteria), the other light-emitting diode lights up green.

The invention is not restricted to the exemplary embodiment described above. Rather, a person skilled in the art may also derive other variants of the invention therefrom, without departing from the subject matter of the invention. For example, all individual features described in conjunction with the exemplary embodiment are also combinable with one another in a different manner, without departing from the subject matter of the invention.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims can, alternatively, be made to depend in the alternative from any preceding or following claim, whether independent or dependent, and that such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A sensor instrument for examining a mucous membrane of an esophagus, a stomach and a duodenum of a patient to see whether the mucous membrane is infected with bacteria, the sensor instrument comprising:
an assembly comprising:
an evaluation unit; and
a catheter probe comprising an ammonia-sensitive sensor and a catheter.

2. The sensor instrument of claim 1, wherein the catheter probe is configured for use in an endoscopic work channel.

3. The sensor instrument of claim 1, wherein the catheter probe is detachably connected to the evaluation unit.

4. The sensor instrument of claim 3, wherein the catheter probe is a disposable article.

5. The sensor instrument of claim 1, wherein the assembly is configured as a complete, intrinsically sealed and non-dismountable assembly.

6. The sensor instrument of claim 5, wherein the instrument is a disposable article.

7. The sensor instrument of claim 6, further comprising endoscopic optics.

8. The sensor instrument of claim 1, wherein the evaluation unit is only configured for evaluating signals from the ammonia-sensitive sensor.

9. The sensor instrument of claim 1, wherein the evaluation unit is configured to output an examination report.

10. The sensor instrument of claim 9, wherein the evaluation unit is configured for wireless transmission of the examination report to a terminal.

11. The sensor instrument of claim 10, wherein the evaluation unit includes a wireless receiver and is configured such that, after receiving a reception confirmation from the terminal confirming the reception of the examination report at the terminal, the evaluation unit switches to a standby mode.

12. The sensor instrument of claim 1, wherein the evaluation unit includes an indicator with two indicator values, wherein one indicator value of the two indicator values indicates the presence of Helicobacter pylori bacteria, and the other indicator value of the two indicator values indicates the non-presence of Helicobacter pylori bacteria.

13. The sensor instrument of claim 1, wherein the evaluation unit is configured for an acoustic signal output with two output values, wherein one output value of the two output values symbolizes the presence of Helicobacter pylori bacteria, and the other output value of the two output values symbolizes the non-presence of Helicobacter pylori bacteria.

14. The sensor instrument of claim 1, wherein the evaluation unit is configured for a signal output of a signal, and
wherein the signal signals an active measurement.

15. The sensor instrument of claim 2, wherein the assembly is configured as a complete, intrinsically sealed and non-dismountable assembly.

16. The sensor instrument of claim 15, wherein the instrument is a disposable article.

17. The sensor instrument of claim 6, further comprising endoscopic optics.
